(19) United States
(12) Patent Application Publication
(10) Pub. No.: US 2010/0125196 A1
(43) Pub. Date: May 20, 2010

(54) Ultrasonic Diagnostic Apparatus And Method For Generating Commands In Ultrasonic Diagnostic Apparatus

(76) Inventors: Jong Min Park, Gunpo-si (KR); Young Seuk Song, Seoul (KR)

Correspondence Address: JONES DAY, 222 EAST 41ST ST, NEW YORK, NY, 10017, US

(21) Appl. No.: 12/422,194

(22) Filed: Apr. 10, 2009

(30) Foreign Application Priority Data
Nov. 17, 2008 (KR) 10-2008-0114017

(51) Int. Cl. A61B 8/00; G06F 3/00

(52) U.S. Cl. 600/437; 715/762

(57) ABSTRACT

An ultrasonic diagnostic apparatus including: a touch panel unit to sense a contact; a pattern identification unit to identify a pattern of the sensed contact; and a command generation unit to generate a command signal corresponding to the identified pattern, as a response to the contact is provided.

### CROSS-REFERENCE TO RELATED APPLICATION

This application claims the benefit of Korean Patent Application No. 10-2008-0114017, filed on Nov. 17, 2008, in the Korean Intellectual Property Office, the entire contents of which are incorporated herein by reference.

### BACKGROUND

1. Field of the Invention

The present invention relates to an ultrasonic diagnostic apparatus that may analyze a pattern of a contact, sensed via a touch panel, and thereby may operate according to a command signal corresponding to the pattern, and a method of generating the command signal at the ultrasonic diagnostic apparatus.

2. Description of the Related Art

An ultrasonic diagnostic apparatus may be one of representative medical equipments. Basically, the ultrasonic diagnostic apparatus may periodically convert, to electrical signals, information carried in ultrasonic waves that are emitted towards a human body and then are returned to the ultrasonic diagnostic apparatus due to a piezoelectric effect of a probe, and may display the converted electrical signals on a screen. Specifically, without a need to incise a diseased part of the human body, the ultrasonic diagnostic apparatus may obtain information associated with the inside of the human body.

The conventional ultrasonic diagnostic apparatus may include various types of functional keys such as a character/number input key to enable a user to input a command and information into a system, a track ball that is an auxiliary input instrument of a host system, a mode selection key to convert a system mode, an encoder to adjust an output signal of the host system or a screen state, a time-gain control (TGC) terminal, and the like.

As described above, in the conventional art, the ultrasonic diagnostic apparatus is controlled using the track ball and thus a separate functional key is required for an operation that is uncontrollable using the track ball. As the separate functional key is added, the size of a control panel of the ultrasonic diagnostic apparatus may increase.

Currently, various types of portable devices with a touch panel are being developed, for example, a notebook computer, a personal digital assistant (PDA), a mobile phone, and the like. A user of the portable device may contact with the touch panel using a pen or a finger to perform pre-registered operations and to thereby readily control the portable device. Specifically, the portable device may enhance a utility for an existing limited space, using the touch panel.

Accordingly, there is a need for a technology that may generate a command signal to operate an ultrasonic diagnostic apparatus using a multi-touch panel instead of using a track ball with a relatively large space limitation.

### SUMMARY

An aspect of the present invention provides an ultrasonic diagnostic apparatus that may analyze a pattern of a contact, sensed via a touch panel, and thereby may operate according to a command signal corresponding to the analyzed pattern, and a method of generating the command signal at the ultrasonic diagnostic apparatus.

Another aspect of the present invention also provides an ultrasonic diagnostic apparatus that may readily generate a command signal capable of operating the ultrasonic diagnostic apparatus using a multi-touch panel, instead of using a track ball with a relatively large space limitation, and a method of generating the command signal at the ultrasonic diagnostic apparatus.

Another aspect of the present invention also provides an ultrasonic diagnostic apparatus that may generate a command signal by further considering a number of contacts, when a plurality of contacts are identified as the same pattern within a predetermined period of time, that is, when multiple touches are sensed via a touch panel, and thereby may perform more various operations, and a method of generating the command signal at the ultrasonic diagnostic apparatus.

Another aspect of the present invention also provides an ultrasonic diagnostic apparatus that may register a command signal, retrieved according to a user's selection, in correspondence to a pattern of a contact and thereby enables the user to more readily remember the pattern matching the command signal, and a method of generating the command signal at the ultrasonic diagnostic apparatus.

Another aspect of the present invention also provides an ultrasonic diagnostic apparatus that may readily generate a command signal capable of operating the ultrasonic diagnostic apparatus using a touch panel and thereby may reduce a size of a control panel to achieve a miniature and a lightness of the ultrasonic diagnostic apparatus, and enables a user to intuitively simply control the ultrasonic diagnostic apparatus via a contact, and thereby may enhance a manipulation ability, and a method of generating the command signal at the ultrasonic diagnostic apparatus.

According to an aspect of the present invention, there is provided an ultrasonic diagnostic apparatus including: a touch panel unit to sense a contact; a pattern identification unit to identify a pattern of the sensed contact; and a command generation unit to generate a command signal corresponding to the identified pattern, as a response to the contact.

According to another aspect of the present invention, there is provided a method of generating a command signal at an ultrasonic diagnostic apparatus, the method including: sensing a contact; identifying a pattern of the sensed contact; and generating a command signal corresponding to the identified pattern, as a response to the contact.

Additional aspects, features, and/or advantages of the invention will be set forth in part in the description which follows and, in part, will be apparent from the description, or may be learned by practice of the invention.

### Effect

According to embodiments of the present invention, it is possible to readily generate a command signal capable of operating the ultrasonic diagnostic apparatus using a multi-touch panel, instead of using a track ball with a relatively large space limitation.

Also, according to embodiments of the present invention, when a plurality of contacts are identified as the same pattern within a predetermined period of time, that is, when multiple touches are sensed via a touch panel, it is possible to generate a command signal by further considering a number of contacts and thereby perform more various operations.

Also, according to embodiments of the present invention, it is possible to register a command signal, retrieved according to a user's selection, in correspondence to a pattern of a contact. Through this, the user may more readily remember the pattern matching the command signal.

Also, according to embodiments of the present invention, it is possible to readily generate a command signal capable of operating the ultrasonic diagnostic apparatus using a touch panel. Through this, since a size of a control panel may be reduced, it is possible to achieve a miniature and a lightness of an ultrasonic diagnostic apparatus. Also, since a user may intuitively simply control the ultrasonic diagnostic apparatus via a contact, it is possible to enhance a manipulation ability.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating a configuration of an ultrasonic diagnostic apparatus according to an embodiment of the present invention;

FIGS. 2A and 2B illustrate examples of a menu list to register, in a database, a command signal in correspondence to a pattern at an ultrasonic diagnostic apparatus according to an embodiment of the present invention;

FIGS. 3A through 3C illustrate examples of a menu list to register, in a database, a command signal in correspondence to a pattern at an ultrasonic diagnostic apparatus according to another embodiment of the present invention;

FIGS. 4A and 4B illustrate examples of a command signal registered by further considering a number of contacts included in a contact group as a plurality of contacts are sensed at an ultrasonic diagnostic apparatus according to an embodiment of the present invention; and

FIG. 5 is a flowchart illustrating a method of generating a command signal at an ultrasonic diagnostic apparatus according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

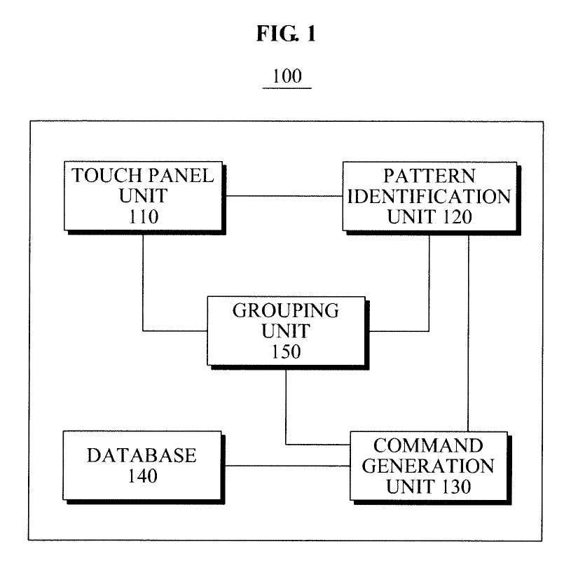

FIG. 1 is a block diagram illustrating a configuration of an ultrasonic diagnostic apparatus 100 according to an embodiment of the present invention.

The ultrasonic diagnostic apparatus 100 may include a touch panel unit 110, a pattern identification unit 120, a command generation unit 130, a database 140, and a grouping unit 150.

The ultrasonic diagnostic apparatus 100 may sense a contact via the touch panel unit 110 and identify a pattern of the sensed contact via the pattern identification unit 120.

Here, the touch panel unit 110 denotes an input device or an interface of the ultrasonic diagnostic apparatus 100. The touch panel unit 110 may sense the contact, caused by a pen or a finger, to determine an input of the ultrasonic diagnostic apparatus 100.

The touch panel unit 110 may be embodied into various types, for example, a touch screen, a touch sensor, and the like, that are included in a display device such as a liquid crystal display (LCD).

Here, the pattern may denote an operation that is performed in a state where the sensed contact is maintained. The pattern identification unit 120 may identify the pattern of the sensed contact as any one of a click, a drag, an extend, a gather, and a rotate.

For example, when an operation of pressing a predetermined point, or an operation of pressing and then releasing the predetermined point is performed, the pattern identification unit 120 may identify the pattern of the contact as a ‘click’. Also, when an operation of dragging the predetermined point while maintaining the contact is performed, the pattern identification unit 120 may identify the pattern of the contact as a ‘drag’. Also, when an operation of rotating the predetermined point while pressing the point to maintain the contact is performed, the pattern identification unit 120 may identify the pattern of the contact as a ‘rotate’. Also, when an operation of sensing a plurality of contacts and extending a distance between the plurality of contacts is performed, the pattern identification unit 120 may identify the pattern of the contact as an ‘extend’. Also, when an operating of sensing the plurality of contacts and narrowing the distance between the plurality of contacts is performed, the pattern identification unit 120 may identify the pattern of the contact as a ‘gather’.

The command generation unit 130 may generate a command signal corresponding to the identified pattern, as a response to the contact.

Specifically, as the contact is sensed, the command generation unit 130 may generate the command signal corresponding to the pattern of the contact and thereby enable the ultrasonic diagnostic apparatus 100 to operate according to the command signal. Accordingly, the ultrasonic diagnostic apparatus 100 may be intuitively simply operated via the contact and thus it is possible to efficiently enhance a manipulation ability.

For example, when the pattern is identified as the above ‘click’, the command generation unit 130 may generate, as the response to the contact, the command signal associated with at least one of a set, an exist and a change.

Also, when the pattern is identified as the above ‘drag’, the command generation unit 130 may generate, as the response to the contact, a command signal associated with at least one of a pointer movement, a region of interest (ROI) box movement, an image movement, a forward movement, a backward movement, an increase in a screen brightness, and a decreases in the screen brightness.

Also, when the pattern is identified as the above ‘extend’, the command generation unit 130 may generate, as the response to the contact, a command signal associated with an image size enlargement or a ROI box size enlargement

Also, when the pattern is identified as the above ‘gather’, the command generation unit 130 may generate, as the response to the contact, a command signal associated with an image size reduction or an ROI box size reduction.

Also, when the pattern is identified as the above ‘rotate’, the command generation unit 130 may generate, as the response to the contact, a command signal associated with an image rotation or an indicator rotation.

According to an embodiment of the present invention, it is possible to readily generate a command signal capable of operating an ultrasonic diagnostic apparatus using a multi-touch panel, instead of using a track ball with a relatively large space limitation.

Also, according to an embodiment of the present invention, it is possible to readily generate a command signal capable of operating an ultrasonic diagnostic apparatus using a touch panel. Through this, since a size of a control panel is reduced, it is possible to achieve a miniature and a lightness of the ultrasonic diagnostic apparatus.

Here, the term ‘ROI box’ may be a region that is designated by a user, as an ROI, in an image, for example, an ultrasound image displayed on a screen of the ultrasonic diagnostic apparatus 100.

Also, the ultrasonic diagnostic apparatus 100 may maintain the database 140 to register a command signal, retrieved according to the user's selection on a menu list, in correspondence to a pattern associated with the menu list. The command generation unit 130 may generate the command signal that is registered in the database 140 in correspondence to the identified pattern.

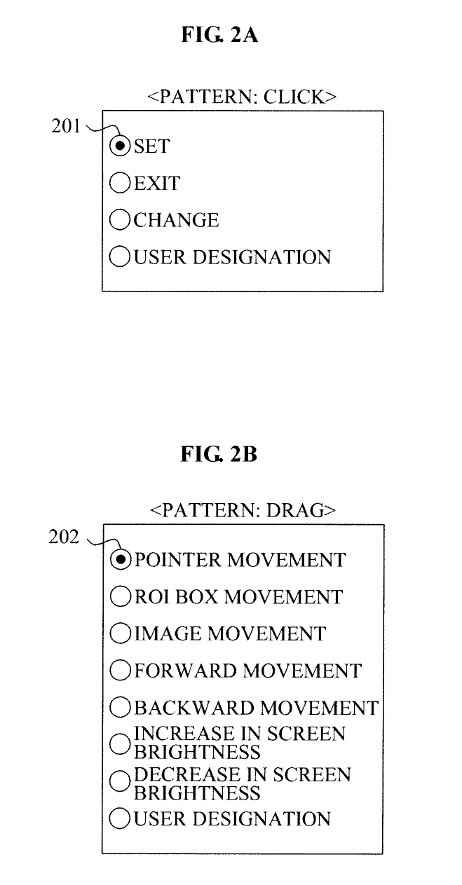

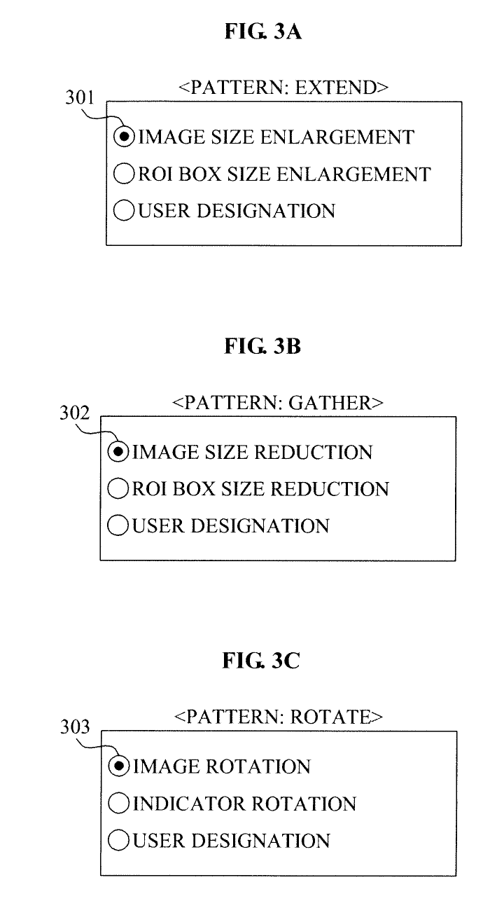

For example, according to an embodiment of the present invention, the ultrasonic diagnostic apparatus 100 may register, in the database 140 and in correspondence to each pattern, a command signal that is selected by the user in menu lists shown in FIGS. 2 through 3.

FIGS. 2A and 2B illustrate examples of a menu list to register, in a database, a command signal in correspondence to a pattern at an ultrasonic diagnostic apparatus according to an embodiment of the present invention.

FIG. 2A illustrates a menu list to register a command signal associated with a pattern ‘click’.

The menu list may include command signals ‘set’, ‘exist’, ‘change’, ‘user designation’, and the like, that may be registered in correspondence to the pattern ‘click’. According to an embodiment of the present invention, the ultrasonic diagnostic apparatus 100 of FIG. 1 may register, in the database 140 and in correspondence to the pattern ‘click’, a command signal ‘set’ 201 that is selected by the user in the menu list. Also, when the command signal ‘user designation’ is selected in the menu list in correspondence to the pattern ‘click’, the ultrasonic diagnostic apparatus 100 may register, in the database 140 and in correspondence to the pattern ‘click’, a new command signal that is not included in the menu list.

When a contact is sensed and a pattern of the contact is identified as the ‘click’, the command generation unit 130 may generate, as a response to the contact, the command signal ‘set’ that is registered in the database 140 in correspondence to the pattern ‘click’. The ultrasonic diagnostic apparatus 100 may perform setting according to the command signal ‘set’.

FIG. 2B illustrates a menu list to register a command signal associated with a pattern ‘drag’.

The menu list may include command signals ‘pointer movement’, ‘ROI box movement’, ‘image movement’, ‘forward movement’, ‘backward movement’, ‘increase in a screen brightness’, ‘decreases in the screen brightness’, ‘user designation’, and the like, that may be registered in correspondence to the pattern ‘drag’. According to an embodiment of the present invention, the ultrasonic diagnostic apparatus 100 of FIG. 1 may register, in the database 140 and in correspondence to the pattern ‘drag’, a command signal ‘pointer movement’ 202 that is selected by the user in the menu list. Also, when the command signal ‘user designation’ is selected in the menu list in correspondence to the pattern ‘drag’, the ultrasonic diagnostic apparatus 100 may register, in the database 140 and in correspondence to the pattern ‘drag’, a new command signal that is not included in the menu list.

When a contact is sensed and a pattern of the contact is identified as the ‘drag’, the command generation unit 130 may generate, as a response to the contact, the command signal ‘pointer movement’ that is registered in the database 140 in correspondence to the pattern ‘drag’. The ultrasonic diagnostic apparatus 100 may move a pointer according to the command signal ‘pointer movement’.

FIGS. 3A through 3C illustrate examples of a menu list to register, in a database, a command signal in correspondence to a pattern at an ultrasonic diagnostic apparatus according to an embodiment of the present invention.

FIG. 3A illustrates a menu list to register a command signal associated with a pattern ‘extend’.

The menu list may include command signals ‘image size enlargement’, ‘ROI box size enlargement’, ‘user designation’, and the like, that may be registered in correspondence to the pattern ‘extend’. According to an embodiment of the present invention, the ultrasonic diagnostic apparatus 100 of FIG. 1 may register, in the database 140 and in correspondence to the pattern ‘extend’, a command signal ‘image size enlargement’ 301 that is selected by the user in the menu list. Also, when the command signal ‘user designation’ is selected in the menu list in correspondence to the pattern ‘extend’, the ultrasonic diagnostic apparatus 100 may register, in the database 140 and in correspondence to the pattern ‘extend’, a new command signal that is not included in the menu list.

When a contact is sensed and a pattern of the contact is identified as the ‘extend’, the command generation unit 130 may generate, as a response to the contact, the command signal ‘image size enlargement’ that is registered in the database 140 in correspondence to the pattern ‘extend’. The ultrasonic diagnostic apparatus 100 may enlarge an image size according to the command signal ‘image size enlargement’.

FIG. 3B illustrates a menu list to register a command signal associated with a pattern ‘gather’.

The menu list may include command signals ‘image size reduction’, ‘ROI box size reduction’, ‘user designation’, and the like that may be registered in correspondence to the pattern ‘gather’. According to an embodiment of the present invention, the ultrasonic diagnostic apparatus 100 of FIG. 1 may register, in the database 140 and in correspondence to the pattern ‘gather’, a command signal ‘image size reduction’ 302 that is selected by the user in the menu list. Also, when the command signal ‘user designation’ is selected in the menu list in correspondence to the pattern ‘gather’, the ultrasonic diagnostic apparatus 100 may register, in the database 140 and in correspondence to the pattern ‘gather’, a new command signal that is not included in the menu list.

When a contact is sensed and a pattern of the contact is identified as the ‘gather’, the command generation unit 130 may generate, as a response to the contact, the command signal ‘image size reduction’ that is registered in the database 140 in correspondence to the pattern ‘gather’. The ultrasonic diagnostic apparatus 100 may reduce an image size according to the command signal ‘image size reduction’.

FIG. 3C illustrates a menu list to register a command signal associated with a pattern ‘rotate’.

The menu list may include command signals ‘image rotation’, ‘indicator rotation’, ‘user designation’, and the like, that may be registered in correspondence to the pattern ‘rotate’. According to an embodiment of the present invention, the ultrasonic diagnostic apparatus 100 of FIG. 1 may register, in the database 140 and in correspondence to the pattern ‘rotate’, the command signal ‘image rotation’ 303 that is selected by the user in the menu list. Also, when the command signal ‘user designation’ is selected in the menu list in correspondence to the pattern ‘rotate’, the ultrasonic diagnostic apparatus 100 may register, in the database 140 and in correspondence to the pattern ‘rotate’, a new command signal that is not included in the menu list.

When a contact is sensed and a pattern of the contact is identified as the ‘rotate’, the command generation unit 130 may generate, as a response to the contact, the command signal ‘image rotation’ that is registered in the database 140 in correspondence to the pattern ‘rotate’. The ultrasonic diagnostic apparatus 100 may rotate an image according to the command signal ‘image rotation’.

As described above, according to an embodiment of the present invention, a command signal retrieved according to a user's selection may be registered in correspondence to a pattern of a contact and thus the user may more readily remember and use the pattern matching the command signal.

Also, the grouping unit 150 may group, into a contact group, a plurality of contacts that are sensed within a predetermined period of time and are identified as the same pattern. The command generation unit 130 may generate the command signal by further considering a number of contacts that are included in the contact group.

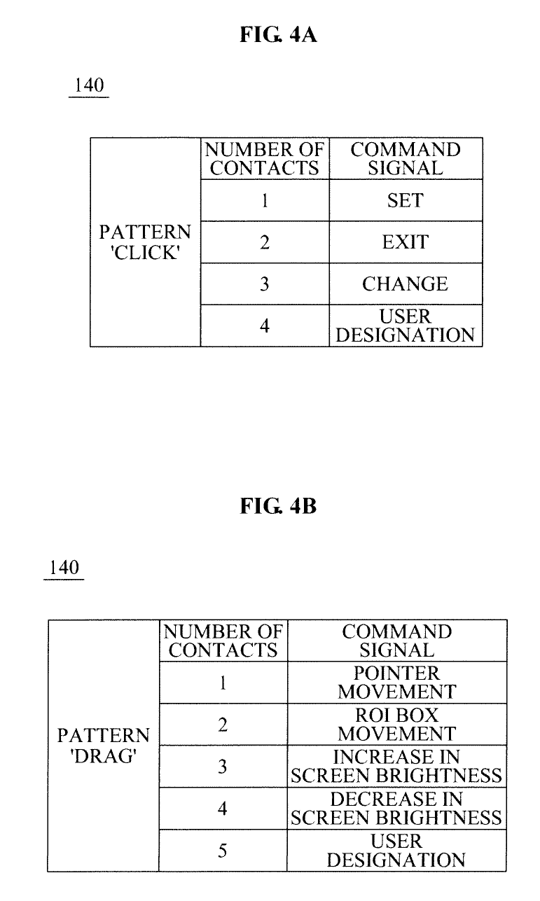

For example, as shown in FIGS. 4A and 4B, the database 140 may register a command signal corresponding to a pattern of a contact by considering a number of contacts. The command generation unit 130 may generate a command signal corresponding to a ‘number of contacts’ included in a contact group, among command signals corresponding to the pattern in the database 140.

FIGS. 4A and 4B illustrate examples of a command signal registered by further considering a number of contacts included in a contact group as a plurality of contacts are sensed at an ultrasonic diagnostic apparatus according to an embodiment of the present invention.

FIG. 4A illustrates an example of the database 140 that registers command signals corresponding to a pattern ‘click’ by further considering a number of contacts.

Specifically, the database 140 may register a command signal ‘set’, among the command signals corresponding to the pattern ‘click’, in further correspondence to the number of contacts, ‘1’. Also, the database 140 may register a command signal ‘exit’, among the command signals corresponding to the pattern ‘click’, in further correspondence to the number of contacts, ‘2’. Also, the database 140 may register a command signal ‘change’, among the command signals corresponding to the pattern ‘click’, in further correspondence to the number of contacts, ‘3’. Also, the database 140 may register a command signal ‘user designation’, among the command signals corresponding to the pattern ‘click’, in further correspondence to the number of contacts, ‘4’.

Accordingly, when the plurality of contacts that are sensed within the predetermined period of time and are identified as the same pattern, for example, the pattern ‘click’, are grouped into a single contact group, the command generation unit 130 may verify the number of contacts included in the contact group, for example, ‘2’, and then generate the command signal, for example, ‘exit’ further corresponding to the verified number of contacts. The ultrasonic diagnostic apparatus 100 may be terminated according to the command signal ‘exit’.

FIG. 4B illustrates an example of the database 140 that registers command signals corresponding to a pattern ‘drag’ by further considering a number of contacts.

Specifically, the database 140 may register a command signal ‘pointer movement’, among the command signals corresponding to the pattern ‘drag’, in further correspondence to the number of contacts, ‘1’. Also, the database 140 may register a command signal ‘ROI box movement’, among the command signals corresponding to the pattern ‘drag’, in further correspondence to the number of contacts, ‘2’. Also, the database 140 may register a command signal ‘increase in a screen brightness’, among the command signals corresponding to the pattern ‘drag’, in further correspondence to the number of contacts, ‘3’. Also, the database 140 may register a command signal ‘decrease in a screen brightness’, among the command signals corresponding to the pattern ‘drag’, in further correspondence to the number of contacts, ‘4’. Also, the database 140 may register a command signal ‘user designation’, among the command signals corresponding to the pattern ‘drag’, in further correspondence to the number of contacts, ‘5’.

Accordingly, when the plurality of contacts that are sensed within the predetermined period of time and are identified as the same pattern, for example, the pattern ‘drag’, are grouped into a single contact group, the command generation unit 130 may verify the number of contacts included in the contact group, for example, ‘3’, and then generate the command signal, for example, ‘increase in the screen brightness’ further corresponding to the verified number of contacts. The ultrasonic diagnostic apparatus 100 may increase the screen brightness according to the command signal ‘increase in the screen brightness’.

As described above, according to an embodiment of the present invention, when the plurality of contacts are identified as the same pattern within a predetermined period of time, that is, when multiple touches are sensed in a touch panel, it is possible to generate a command signal by further considering a number of contacts. Through this, the ultrasonic diagnostic apparatus 100 may be more variously operated.

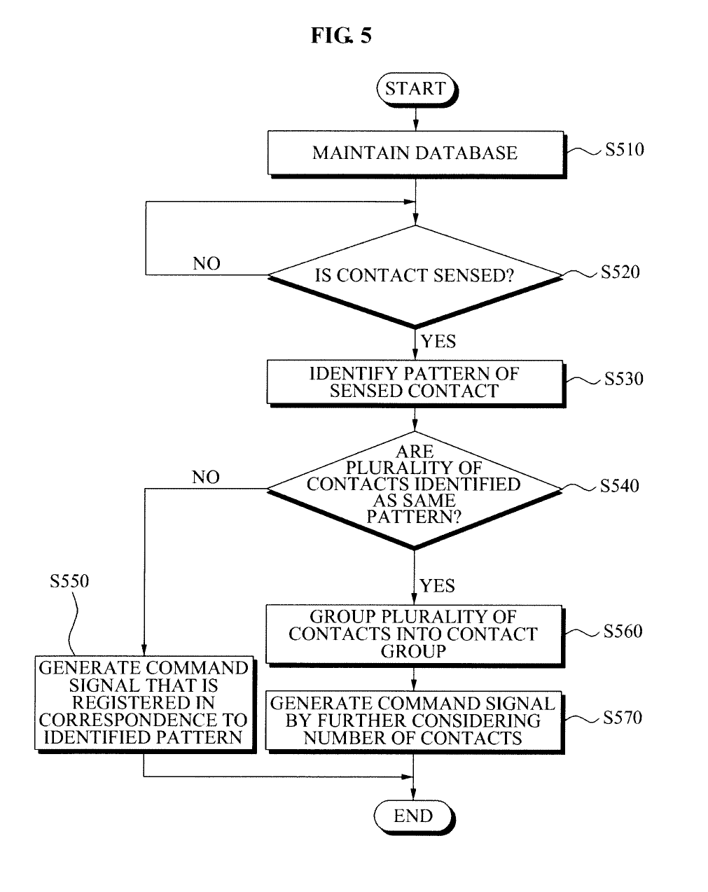

FIG. 5 is a flowchart illustrating a method of generating a command signal at an ultrasonic diagnostic apparatus according to an embodiment of the present invention.

The command signal generating method at the ultrasonic diagnostic apparatus according to an embodiment of the present invention may be performed by the ultrasonic diagnostic apparatus 100 of FIG. 1. Hereinafter, the method will be described with reference to FIGS. 1 and 5.

In operation S510, the ultrasonic diagnostic apparatus 100 may maintain the database 140 to register a command signal, retrieved according to a user's selection on a menu list, in correspondence to a pattern associated with the menu list.

For example, the ultrasonic diagnostic apparatus 100 may register, in the database 140 and in correspondence to each pattern, a command signal that is selected by a user in menu lists as shown in FIGS. 2 through 3. Accordingly, the user may more readily remember and use the pattern matching the command signal.

FIG. 2A illustrates a menu list to register a command signal associated with a pattern ‘click’.

The menu list may include command signals ‘set’, ‘exist’, ‘change’, ‘user designation’, and the like, that may be registered in correspondence to the pattern ‘click’. According to an embodiment of the present invention, the ultrasonic diagnostic apparatus 100 of FIG. 1 may register, in the database 140 and in correspondence to the pattern ‘click’, the command signal ‘set’ 201 that is selected by the user in the menu list. Also, when the command signal ‘user designation’ is selected in the menu list in correspondence to the pattern ‘click’, the ultrasonic diagnostic apparatus 100 may register, in the database 140 and in correspondence to the pattern ‘click’, a new command signal that is not included in the menu list.

FIG. 2B illustrates a menu list to register a command signal associated with a pattern ‘drag’.

The menu list may include command signals ‘pointer movement’, ‘ROI box movement’, ‘image movement’, ‘forward movement’, ‘backward movement’, ‘increase in a screen brightness’, ‘decreases in the screen brightness’, ‘user designation’, and the like, that may be registered in correspondence to the pattern ‘drag’. According to an embodiment of the present invention, the ultrasonic diagnostic apparatus 100 of FIG. 1 may register, in the database 140 in correspondence to the pattern ‘drag’, the command signal ‘pointer movement’ 202 that is selected by the user in the menu list. Also, when the command signal ‘user designation’ is selected in the menu list in correspondence to the pattern ‘drag’, the ultrasonic diagnostic apparatus 100 may register, in the database 140 and in correspondence to the pattern ‘drag’, a new command signal that is not included in the menu list.

FIG. 3A illustrates a menu list to register a command signal associated with a pattern ‘extend’.

The menu list may include command signals ‘image size enlargement’, ‘ROI box size enlargement’, ‘user designation’, and the like, that may be registered in correspondence to the pattern ‘extend’. According to an embodiment of the present invention, the ultrasonic diagnostic apparatus 100 of FIG. 1 may register, in the database 140 and in correspondence to the pattern ‘extend’, the command signal ‘image size enlargement’ 301 that is selected by the user in the menu list. Also, when the command signal ‘user designation’ is selected in the menu list in correspondence to the pattern ‘extend’, the ultrasonic diagnostic apparatus 100 may register, in the database 140 and in correspondence to the pattern ‘extend’, a new command signal that is not included in the menu list.

FIG. 3B illustrates a menu list to register a command signal associated with a pattern ‘gather’.

The menu list may include command signals ‘image size reduction’, ‘ROI box size reduction’, ‘user designation’, and the like that may be registered in correspondence to the pattern ‘gather’. According to an embodiment of the present invention, the ultrasonic diagnostic apparatus 100 of FIG. 1 may register, in the database 140 and in correspondence to the pattern ‘gather’, the command signal ‘image size reduction’ 302 that is selected by the user in the menu list. Also, when the command signal ‘user designation’ is selected in the menu list in correspondence to the pattern ‘gather’, the ultrasonic diagnostic apparatus 100 may register, in the database 140 and in correspondence to the pattern ‘gather’, a new command signal that is not included in the menu list.

FIG. 3C illustrates a menu list to register a command signal associated with a pattern ‘rotate’.

The menu list may include command signals ‘image rotation’, ‘indicator rotation’, ‘user designation’, and the like, that may be registered in correspondence to the pattern ‘rotate’. According to an embodiment of the present invention, the ultrasonic diagnostic apparatus 100 of FIG. 1 may register, in the database 140 and in correspondence to the pattern ‘rotate’, the command signal ‘image rotation’ 303 that is selected by the user in the menu list. Also, when the command signal ‘user designation’ is selected in the menu list in correspondence to the pattern ‘rotate’, the ultrasonic diagnostic apparatus 100 may register, in the database 140 and in correspondence to the pattern ‘rotate’, a new command signal that is not included in the menu list.

Also, as shown in FIGS. 4A and 4B, the database 140 may register a command signal corresponding to a pattern of a contact by considering a number of contacts.

FIG. 4A illustrates an example of the database 140 that registers command signals corresponding to a pattern ‘click’ by further considering a number of contacts.

Specifically, the database 140 may register a command signal ‘set’, among the command signals corresponding to the pattern ‘click’, in further correspondence to the number of contacts, ‘1’. Also, the database 140 may register a command signal ‘exit’, among the command signals corresponding to the pattern ‘click’, in further correspondence to the number of contacts, ‘2’. Also, the database 140 may register a command signal ‘change’, among the command signals corresponding to the pattern ‘click’, in further correspondence to the number of contacts, ‘3’. Also, the database 140 may register a command signal ‘user designation’, among the command signals corresponding to the pattern ‘click’, in further correspondence to the number of contacts, ‘4’.

FIG. 4B illustrates an example of the database 140 that registers command signals corresponding to a pattern ‘drag’ by further considering a number of contacts.

Specifically, the database 140 may register a command signal ‘pointer movement’, among the command signals corresponding to the pattern ‘drag’, in further correspondence to the number of contacts, ‘1’. Also, the database 140 may register a command signal ‘ROI box movement’, among the command signals corresponding to the pattern ‘drag’, in further correspondence to the number of contacts, ‘2’. Also, in the database 140 may register a command signal ‘increase in a screen brightness’, among the command signals corresponding to the pattern ‘drag’, in further correspondence to the number of contacts, ‘3’. Also, the database 140 may register a command signal ‘decrease in a screen brightness’, among the command signals corresponding to the pattern ‘drag’, in further correspondence to the number of contacts, ‘4’. Also, the database 140 may register a command signal ‘user designation’, among the command signals corresponding to the pattern ‘drag’, in further correspondence to the number of contacts, ‘5’.

In operation S520, the ultrasonic diagnostic apparatus 100 may determine whether the contact is sensed.

As described above, the touch panel unit 110 denotes an input device or an interface of the ultrasonic diagnostic apparatus 100. The touch panel unit 110 may sense the contact, caused by a pen or a finger, to determine an input of the ultrasonic diagnostic apparatus 100.

The touch panel unit 110 may be embodied into various types, for example, a touch screen, a touch sensor, and the like, that are included in a display device such as an LCD.

When it is determined the contact is not sensed in operation S520, the ultrasonic diagnostic apparatus 100 may return to operation S510 and repeat operation S520.

Conversely, when it is determined the contact is sensed in operation S520, the ultrasonic diagnostic apparatus 100 may identify a pattern of the sensed contact in operation S530.

Here, the pattern may denote an operation that is performed in a state where the sensed contact is maintained. The pattern identification unit 120 may identify the pattern of the sensed contact as any one of a click, a drag, an extend, a gather, and a rotate.

For example, when an operation of pressing a predetermined point, or an operation of pressing and then releasing the predetermined point is performed, the pattern identification unit 120 may identify the pattern of the contact as a ‘click’. Also, when an operation of dragging the predetermined point while maintaining the contact is performed, the pattern identification unit 120 may identify the pattern of the contact as a ‘drag’. Also, when an operation of rotating the predetermined point while pressing the point to maintain the contact is performed, the pattern identification unit 120 may identify the pattern of the contact as a ‘rotate’. Also, when an operation of sensing a plurality of contacts and extending a distance between the plurality of contacts is performed, the pattern identification unit 120 may identify the pattern of the contact as an ‘extend’. Also, when an operating of sensing the plurality of contacts and narrowing the distance between the plurality of contacts is performed, the pattern identification unit 120 may identify the pattern of the contact as a ‘gather’.

In operation S540, the ultrasonic diagnostic apparatus 100 may determine whether a plurality of contacts that are sensed within a predetermined period of time and are identified as the same pattern are sensed.

When it is determined the plurality of contacts are not sensed in operation S540, the ultrasonic diagnostic apparatus 100 may generate, as a response to the contact, a command signal to be registered in the database 140 in correspondence to the identified pattern in operation S550.

Specifically, as the contact is sensed, the command generation unit 130 may generate the command signal corresponding to the pattern of the contact and thereby enable the ultrasonic diagnostic apparatus 100 to operate according to the command signal. Accordingly, the ultrasonic diagnostic apparatus 100 may be intuitively simply operated via the contact and thus it is possible to efficiently enhance a manipulation ability.

For example, when the pattern of the sensed contact is identified as the ‘click’, the command generation unit 130 may generate, as the response to the contact, a command signal ‘set’ that is registered in the database 140 in correspondence to the identified pattern ‘click’. The ultrasonic diagnostic apparatus 100 may perform setting according to the command signal ‘set’.

Also, when the pattern of the sensed contact is identified as the ‘drag’, the image generation unit 130 may generate, as the response to the contact, a command signal ‘pointer movement’ that is registered in the database 140 in correspondence to the identified pattern ‘drag’. The ultrasonic diagnostic apparatus 100 may move a pointer according to the command signal ‘pointer movement’.

Also, when the pattern of the sensed contact is identified as the ‘extend’, the command generation unit 130 may generate, as the response to the contact, a command signal ‘image size enlargement’ that is registered in the database 140 in correspondence to the identified pattern ‘extend. The ultrasonic diagnostic apparatus 100 may enlarge an image size according to the command signal ‘image size enlargement’.

Also, when the pattern of the sensed contact is identified as the ‘gather’, the command generation unit 130 may generate, as the response to the contact, a command signal ‘image size reduction’ that is registered in the database 140 in correspondence to the identified pattern ‘gather’. The ultrasonic diagnostic apparatus 100 may reduce the image size according to the command signal ‘image size reduction’.

Also, when the pattern of the sensed contact is identified as the ‘rotate’, the command generation unit 130 may generate, as the response to the contact, a command signal ‘image rotation’ that is registered in the database 140 in correspondence to the identified pattern ‘rotate’. The ultrasonic diagnostic apparatus 100 may rotate an image according to the command signal ‘image size reduction’.

Therefore, according to an embodiment of the present invention, it is possible to readily generate a command signal capable of operating an ultrasonic diagnostic apparatus using a multi-touch panel, instead of using a track ball with a relatively large space limitation.

Also, according to an embodiment of the present invention, it is possible to readily generate a command signal capable of operating an ultrasonic diagnostic apparatus, using a touch panel. Through this, since a size of a control panel is reduced, it is possible to achieve a miniature and a lightness of the ultrasonic diagnostic apparatus.

Conversely, when it is determined the plurality of contacts are sensed in operation S540, the ultrasonic diagnostic apparatus 100 may group, into a contact group, the plurality of contacts that are sensed within the predetermined period of time and are identified as the same pattern in operation S560.

In operation S570, when generating a command signal to be registered in the database 140 in correspondence to the identified pattern, as a response to the contact, the ultrasonic diagnostic apparatus 100 may generate the command signal by further considering a number of contacts included in the contact group.

For example, when the plurality of contacts that are sensed within the predetermined period of time and are identified as the same pattern ‘click’ are grouped into a single contact group, the command generation unit 130 may verify the number of contacts, ‘2’, of the contact group and generate a command signal ‘exit’ further corresponding to the verified number of contacts. The ultrasonic diagnostic apparatus 100 may be terminated according to the command signal ‘exit’.

As another example, when the plurality of contacts that are sensed within the predetermined period of time and are identified as the same pattern ‘drag’ are grouped into a single contact group, the command generation unit 130 may verify the number of contacts, ‘3’, of the contact group and generate a command signal ‘increase in a screen brightness’ further corresponding to the verified number of contacts. The ultrasonic diagnostic apparatus 100 may increase the screen brightness according to the command signal ‘increase in the screen brightness’.

As described above, according to an embodiment of the present invention, when the plurality of contacts are identified as the same pattern within a predetermined period of time, that is, when multiple touches are sensed in a touch panel, it is possible to generate a command signal by further considering a number of contacts. Through this, the ultrasonic diagnostic apparatus 100 may be more variously operated.

The above-described exemplary embodiments of the present invention may be recorded in computer-readable media including program instructions to implement various operations embodied by a computer. The media may also include, alone or in combination with the program instructions, data files, data structures, and the like. Examples of computer-readable media include magnetic media such as hard disks, floppy disks, and magnetic tape; optical media such as CD ROM disks and DVDs; magneto-optical media such as floptical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described exemplary embodiments of the present invention, or vice versa.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. An ultrasonic diagnostic apparatus comprising:
a touch panel unit to sense a contact;
a pattern identification unit to identify a pattern of the sensed contact; and
a command generation unit to generate a command signal corresponding to the identified pattern, as a response to the contact.

2. The ultrasonic diagnostic apparatus of claim 1, further comprising:
a grouping unit to group, into a contact group, a plurality of contacts that are sensed within a predetermined period of time and are identified as the same pattern,
wherein the command generation unit generates the command signal by further considering a number of contacts that are included in the contact group.

3. The ultrasonic diagnostic apparatus of claim 1, further comprising:
a database to register a command signal, retrieved according to a user's selection on a menu list, in correspondence to a pattern associated with the menu list,
wherein the command generation unit generates the command signal that is registered in the database in correspondence to the identified pattern.

4. The ultrasonic diagnostic apparatus of claim 1, wherein, when the pattern is identified as a click, the command generation unit generates, as the response to the contact, a command signal associated with at least one of a set, an exist, and a change.

5. The ultrasonic diagnostic apparatus of claim 1, wherein, when the pattern is identified as a drag, the command generation unit generates, as the response to the contact, a command signal associated with at least one of a pointer movement, a region of interest (ROI) box movement, an image movement, a forward movement, a backward movement, an increase in a screen brightness, and a decreases in the screen brightness.

6. The ultrasonic diagnostic apparatus of claim 1, wherein, when the pattern is identified as an extend, the command generation unit generates, as the response to the contact, a command signal associated with an image size enlargement or a ROI box size enlargement.

7. The ultrasonic diagnostic apparatus of claim 1, wherein, when the pattern is identified as a gather, the command generation unit generates, as the response to the contact, a command signal associated with an image size reduction or an ROI box size reduction.

8. The ultrasonic diagnostic apparatus of claim 1, wherein, when the pattern is identified as a rotate, the command generation unit generates, as the response to the contact, a command signal associated with an image rotation or an indicator rotation.

9. A method of generating a command signal at an ultrasonic diagnostic apparatus, the method comprising:
sensing a contact;
identifying a pattern of the sensed contact; and
generating a command signal corresponding to the identified pattern, as a response to the contact.

10. The method of claim 9, further comprising:
grouping, into a contact group, a plurality of contacts that are sensed within a predetermined period of time and are identified as the same pattern,
wherein the generating comprises generating the command signal by further considering a number of contacts that are included in the contact group.

11. The method of claim 9, further comprising:
maintaining a database to register a command signal, retrieved according to a user's selection on a menu list, in correspondence to a pattern associated with the menu list,
wherein the generating comprises generating the command signal that is registered in the database in correspondence to the identified pattern.

12. The method of claim 9, wherein the identifying comprises identifying, the pattern of the sensed contact as any one of a click, a drag, an extend, a gather, and a rotate.
